(19) United States
(12) Patent Application Publication
(10) Pub. No.: US 2014/0180111 A1
(43) Pub. Date: Jun. 26, 2014

(54) REMOTE CONTROLLED TELEMEDICAL ULTRASONIC DIAGNOSTIC DEVICE

(71) Applicant: IneedMD, Inc., New York, NY (US)

(72) Inventors: Govindan Gopinathan, Ordell, NJ (US); JOSEPH CARVALKO, NORTH PALM BEACH, FL (US); Arthur Tilford, Yorba Linda, CA (US)

(73) Assignee: INEEDMD, INC., New York, NY (US)

(21) Appl. No.: 14/108,455

(22) Filed: Dec. 17, 2013

Related U.S. Application Data
(60) Provisional application No. 61/740,528, filed on Dec. 21, 2012.

(51) Int. Cl. A61B 8/00; A61B 5/00; A61B 8/08; A61B 8/14

(52) U.S. Cl. CPC A61B 8/565; A61B 8/145; A61B 8/4494; A61B 8/463; A61B 8/54; A61B 5/6806; A61B 8/4281; A61B 8/5238
USPC 600/447

(57) ABSTRACT

An apparatus and method for remote medical diagnosis includes using at least one wearable ultrasonic imaging device, more specifically an ultrasonic glove-like apparatus or ultrasonic array containment, for forming three dimensional scanned diagnostic images, transmitting the images to a remote location, and locally or remotely controlling the operation of the ultrasonic imaging.

### CROSS-REFERENCE TO RELATED APPLICATIONS

The application is related to and incorporates by reference the benefit of the earlier filing date, under 35 USC 120 of the provisional patent application Ser. No. 61/740,528 filed Dec. 21, 2012, entitled: A Remote Controlled Telemedical Ultrasonic Diagnostic Device.

### FIELD OF INVENTION

The present invention relates to a device and a method for remote medical diagnosis utilizing an ultrasonic imaging device and a remote control in operative association with a glove-like device worn by a caregiver and applied to a patient's body.

### BACKGROUND

Telemedicine medical care, as provided through electronic communication pathways to patients at locations remote from medical personnel, has been evolving as an efficient means for treatment. However, until now, no portable devices can in real-time (a) ultrasonically create a three dimensional image of organs and bones, hidden beneath flesh, and (b) transmit those images to a remote medical facility. And, further, no portable ultrasonic device exists that in real-time can be employed by relatively unskilled emergency personnel to assist physicians at a distance in diagnosing a medical condition diagnosable through ultrasonic imaging.

Ultrasonic medical devices typically require medical personal that are highly trained in their used and although New Mexico is the only state that regulates diagnostic medical sonographers, certification examinations for sonographers are provided by the American Registry of Diagnostic Medical Sonography, the Cardiovascular Credentialing International and the American Registry of Radiological Technologists. Thus user skill begins at the level of applying the device to the anatomy safely and therefore a problem that needs to be overcome in employing a portable ultrasonic device is that it should be safely operable by relatively unskilled or untrained individuals.

A user of an ultrasonic imaging device moves the device over the affected area of the anatomy, where the user operates the device while looking at a display to determine if the sonogram or ultrasonic image (both having essentially the same meaning) is being acquired satisfactorily. Unskilled users of ultrasonic imaging devices would not have the experience as to where to move the device, and therefore, one requirement for a portable ultrasonic device that may be used by the relatively unskilled is that it be relatively fixed to the anatomy, and not require movement by the user, except for repositioning as may be dictated by a physician at a remote viewing facility. A requirement therefore is that an ultrasonic device be either self-scanning or under local or a remote control of an operator relative to the unit.

The technique of ultrasonic imaging has been widely investigated and is described, for example, by D. E. Robinson in the Proceeding of the Institution of Radio and Electronics Engineers, Australia, Vol. 31, No. 11, pages 385-392, November 1970; “The Application of Ultrasound in Medical Diagnosis.” As pointed out in this article, ultrasonic's may be used to display anatomical cross-sections of organs. Over the past thirty years ultrasonic imaging has proved itself as a diagnostic aid for areas of the abdomen and pregnant uterus, eye, breast, lung, kidney, liver and heart and especially useful where X-rays may be dangerous.

Two-dimensional (2D) ultrasonic probes are necessary to support three-dimensional (3D) electronically produced volume data for many clinical applications. State-of-the-art one-dimensional (1D and 1.5D) probes which electronically scan only in azimuth provide 2D ultrasound images (azimuth and range), which today, are commonly used. Two dimensional probes scan electronically in elevation, as well as azimuth, to provide a 3D data cube (azimuth, elevation and range), which can be processed using image processing software to produce a variety of image formats, such as conventional planar images, planar images at arbitrary scan planes, as well as representations such as surface rendering and orthographic presentations.

Currently available low cost ultrasonic imaging devices are either mechanically scanned devices or one dimensional (1D) phased arrays devices, which contain single rows of parallel elements spaced in the azimuthal direction. Each of these rows of parallel elements can produce a B-scan, i.e., an X-Y image plane or slice at a point along Z-axis, or a depth perpendicular to the surface of a patient's body relative to the face of the transducer. The velocity of sound in human soft tissue is about 1540 m/s. In practice, the transducers are moved over the body area to be examined and the position, of the transducers, is tracked. An image of the artifact's surface as reflected by the ultrasound can be developed by plotting the returns, i.e., the echo position for each transducer position, on a CRT. This is referred to as B mode scanning. While the B-scan can be swept through a volume of tissue and the user can, in principle, visualize the 3D anatomy, such visualization requires significant training and experience. However, transmission of the data to a remote viewing station, where highly trained technicians or doctors can interpret the images, can relax the requirement that the caregiver at the site of the patient also be highly trained.

A C-scan ultrasonic device displays a 2-D image, parallel to the surface of a patient's body, for viewing an organ within the body. Both B-scan and C-Scan systems requires ultrasonic beam-direction and beamforming technology to move the beam from point to point, as well as to provide for focus and depth of field.

A phased array of transmit/receive transducers are capable of moving the beam position in space, without manually moving an acoustic probe or using mechanical scanning device. The term ‘phased array’ originates from the fact that when considering sinusoidal signals such as represented by acoustic waves, a phase shifting or time delay can be introduced at the point of transmission to translate or shift of the phase of the signal.

Acoustic two-dimensional arrays consist of ultrasonically radiating elements arranged in two directions X-Y. If we were to assume that at a 7.5 MHz ultrasonic frequency has a wavelength in tissue of about 0.2 mm, then at 7.5 MHz, an array of 256 ultrasonic transducer elements each separated one wavelength in the×direction and each separated 40 wavelengths in Y direction would span rectangle of about 51 mm in×and 8 mm in Y.

Either B-Scan or C-Scan devices may be embodied in the present invention, both which requires beamforming, which may be achieved using a linear array of acoustic ultrasonic transducers and a beamforming processor that delays, using either analog or digital circuitry, each transducer output by a fixed amount of time. FIGS. 2a-d illustrate the basic principle used to produce a scanning acoustic wavefront or beam of acoustic energy, that is both targeted at a particular location (X, Y, Z in an orthogonal Cartesian coordinate space) within the body and with a particular focus.

U.S. Pat. No. 7,285,094 entitled “3D ultrasonic imaging apparatus and method,” incorporated herein by reference, discusses considerations and solutions for 3D scanning. If one extends a 1D phased array to 2D phased array (of equal size in both directions), two issues arise as pertains to: 1. the prohibitive cost; 2. the frame-time to acquire a 3D volume, which is far greater than the time it takes to acquire a 2D image. By way of example, a linear array with 256 elements extended to two dimensions increases the number of transducers to 256×256 or 65,536 transducers, which by today's level of technology is challenging. It takes longer to acquire the data for a 3D image volume, as compared to the tens of milliseconds needed to acquire a conventional 2D image plane, since there are many more beams needed to interrogate each data point in the volume. At least 128×128=16,384 beams are needed, for each transmit focal range, with about 100 microseconds of two-way time needed for each shot (this assumes a 10 kHz firing rate (pulse repetition rate) and a 7 cm depth). For two focal ranges, this implies an acquisition time of 3.2 seconds, assuming the number of channels available equals the number of elements used in the beamformer. The aforementioned difficulties require practical trade-offs and novel solutions if 2D arrays supporting 3D electronic volume data acquisition are to be an affordable reality.

The ultrasonic system described by the invention herein is not limited to a 1D, 2D or three-dimensional 3D scanning regimen. In the case of a 2D scanning regimen, the user, as will be described be able to view “slices” of images through a zone of interest, thereby reducing the volume of data that requires processing a full 3D volumetric image.

The array elements are usually fed from a single transmit beamformer through a network of the ultrasonic radiating elements referred to as transducers. Referring again to FIG. 2a-d to understand how a beamformed array transmission works, the radiating elements can be thought of as consisting of radiation sources, whose individual contributions are added to the wave front as it progresses towards its intended target in the X, Y, Z space, in order to yield the directional pattern of the device. An array is a group of such elements, and its directional pattern is determined by superimposing the acoustic waves at various phases, achieved in various embodiments by delaying acoustic transmission. Hence, the shape of the beam is determined by the properties of the individual elements, plus the power distribution among the elements (a summation of the amplitudes and phases) and the geometric spatial details of their arrangement in an X-Y plane.

As indicated above, phased arrays contain delay elements, in the feeding function, which serve to provide a scanning ultrasonic beam. By controlling these delay elements, mechanical movement of the beam structure can be simulated as shown in FIG. 2a-d of the prior art and a shown in FIG. 3, an embodiment of the present invention.

An ultrasonic imaging system, such as disclosed in U.S. Pat. No. 7,402,136, incorporated herein by reference, includes an array of transducers, a transmit beamformer, and a receive beamformer to acquire a 3D image of the anatomical volume of interest. The transmit beamformer supplies electrical waveform signals to a 2D transducer array, which in turn produces ultrasonic signals as reflected from internal body structures. Elements in front of the transducer arrays scatter ultrasonic energy back to the transducers, which then generates received electrical signals. The electrical receive signals are delayed for selected times specific to each transducer so that ultrasonic energy scattered from selected zones adds coherently (amplitude and phase consistent with the transmission,) while ultrasonic energy from other zones do not add.

A beamformer utilizing current digital technology is capable of delivering a new point of data at least every 25 nano-seconds. Typically the beamforming delay operation provides delays, by buffering analog to digital (A/D) converted transducer outputs in a digital memory, which vary the read times.

An array processing technique for processing received beamforming signals also allow the beam to be steered, eliminating the requirement that the ultrasonic device be moved over the anatomical volume of interest. The array can remain stationary over an area on the body, the ultrasonic image can be acquired, and then the array moved to another location, where the acquisition process is repeated.

A prior art beamforming ultrasonic system as disclosed in U.S. Pat. No. 6,578,372 B2, entitled “Ultrasound System for the Production of 3-D Images,” incorporated herein by reference, illustrates the principle for scanning or steering the acoustic beam in a plane parallel to a body, referred to as the direction of the X-Y axes, respectively. In a linear phased array, the elements are excited by a single pulse, but with typically less than 1.0 microsecond time differences, that create the “phasing” between adjacent elements, so that the resulting sound pulses add or superimpose the sound wavefront along an X-Y direction and thus the acoustic beam is steered. In addition to steering the beam, the phased array can also focus the beam, in the z-axis direction or in the direction of increasing depth from the surface of the patient's body, by putting curvature in the phase delay pattern. Increased curvature focuses closer to the transducer array, while decreased curvature moves the focus deeper. Delay can also be used with a linear sequenced array to provide focusing. Conversely, upon reception of echoes, delays are used as sampling times of the amplitudes of the electronic signals, which the ultrasonic image produced.

U.S. Pat. No. 7,402,136, entitled “Efficient ultrasonic system for two-dimensional C-scan imaging and related method thereof,” incorporated herein by reference, discusses the problems, such as slowed processing, when a 2D transducer array increases the number of time delay operations, but also the number of physical connections to each transducer complicates the fabrication of the arrays (most commentators believe it is proportional to the square of the transducers in the array as mentioned above.) The '136 patent offers as solution in a system that includes, in part, a 2D transducer array and associated transmit signal for each element, a memory buffer with channels for each element and a receive beamformer. On a time serial, i.e., sequential basis, several times for each line of transducer firing, the contents of each element in a memory buffer are read into the beamformer with different focusing values during each buffer read cycle. In this way, a beamformer algorithm can calculate the beamformed image values for multiple points per line firing cycle or for each line of signals between the transducer array and receive beamformer.

The incorporation of a 2D array, and the ability to scan the internal slices, between zones or the entire volume of a bodily organ (collectively referred hereinafter to “3D images”) using relatively unskilled ultrasonic operators would greatly benefit the medical diagnosis of individuals remotely situated from a medical facility or in emergency situations.

### SUMMARY OF THE INVENTION

The present invention relates to a glove-like article, positioned over a portion of a patient's body for generating ultrasonic signals in response to corresponding characteristics of the patient, and transmitting the diagnostic data to a remote location for remote diagnosis and remote control over the scanning, focusing and selection of the zone of interest within the bodily cavity.

In one embodiment the invention relates to an apparatus either contained in a wearable glove or contained in a holder attached to the body for transmitting a sonogram to a remote location that where an array of ultrasonic transducers are positioned over a portion of the patient's body, and where the transducers are electronically steered and focused for creating a three dimensional sonogram representation of a portion of a patient's anatomy.

In yet another embodiment, the invention includes a method for remote medical diagnosing by medical personnel including fixing an ultrasonic scanning device over a part of a patient anatomy, scanning ultrasonically the anatomy, collecting ultrasonic image data, transmitting the data, viewing the images transmitted. The method further includes (1) initiating the acquisition a subsequent series of ultrasonic images based on the viewing; (2) transmitting control data to alter the ultrasonic scanning device state of one of: X, Y, or Z position; (3) transmitting control data to alter the ultrasonic scanning device state of one of: beam width, beam focus, rate of frame scan or depth of field.

### BRIEF DESCRIPTION OF THE DRAWINGS

FIG. 1a is a diagram of the ultrasonic transducer system according to one embodiment of the present invention;

FIG. 1b is a diagram of the ultrasonic transducer glove according to one embodiment of the present invention;

FIG. 1c is a diagram of the ultrasonic transducer attached to containment plate according to one embodiment of the present invention;

FIG. 2a-d are prior art illustrations of the effects of a beamformer in changing the attitude of the acoustic beam resulting in effectively scanning one or more zones of interest;

FIG. 3 is an isometric view of a glove apparatus of the present invention showing the placement of the associated ultrasonic transducer according to one embodiment of the present invention;

FIG. 4 is a prior art illustration of the effects of a beamformer transducer focusing and sweeping a line array at different depth zones, using time delays associated with each zone;

FIG. 5 is a block diagram of a phased array ultrasonic transducer system according to one embodiment of the present invention;

FIG. 6 is a flow chart illustrating a method for remote controlling the ultrasonic transducer system for medical consultation according to one embodiment of the present invention.

### DETAILED DESCRIPTION OF THE PREFERRED EMBODIMENTS

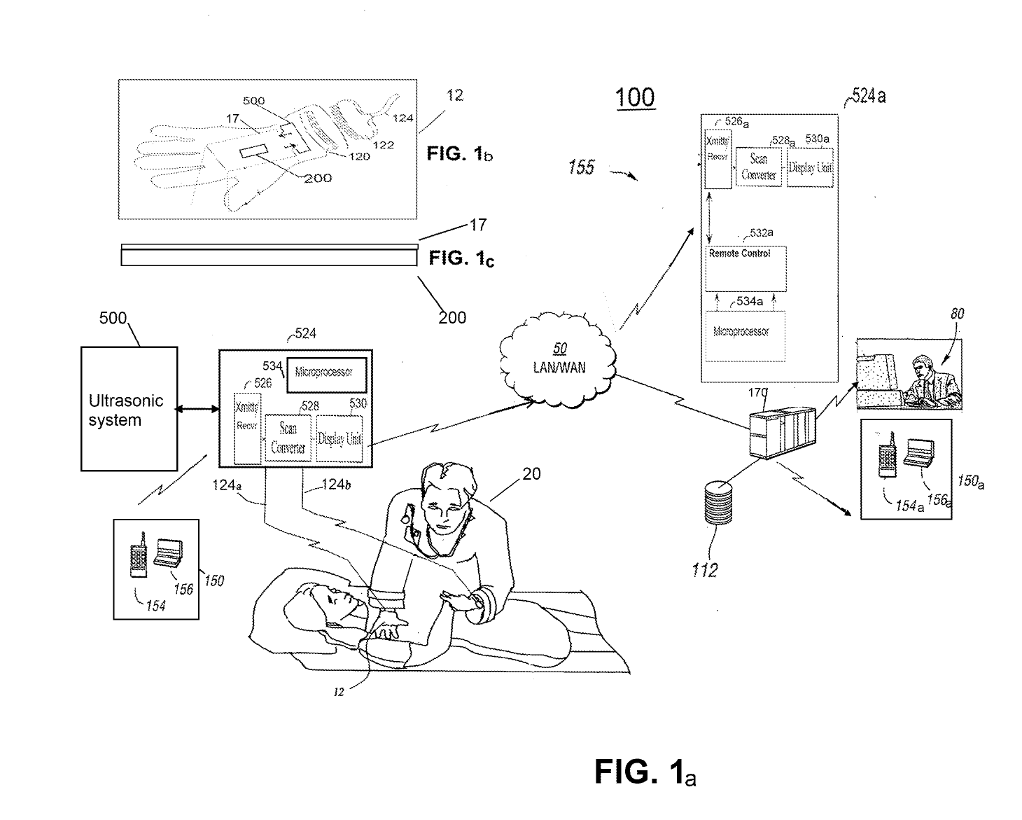

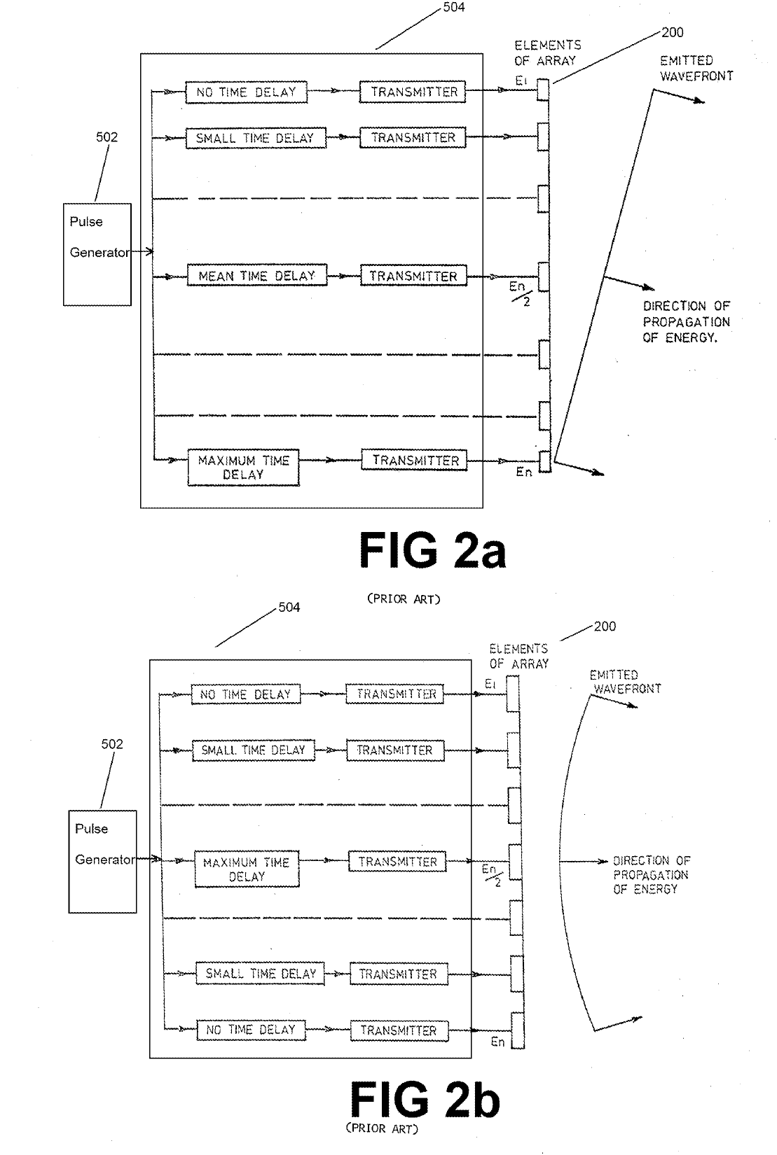

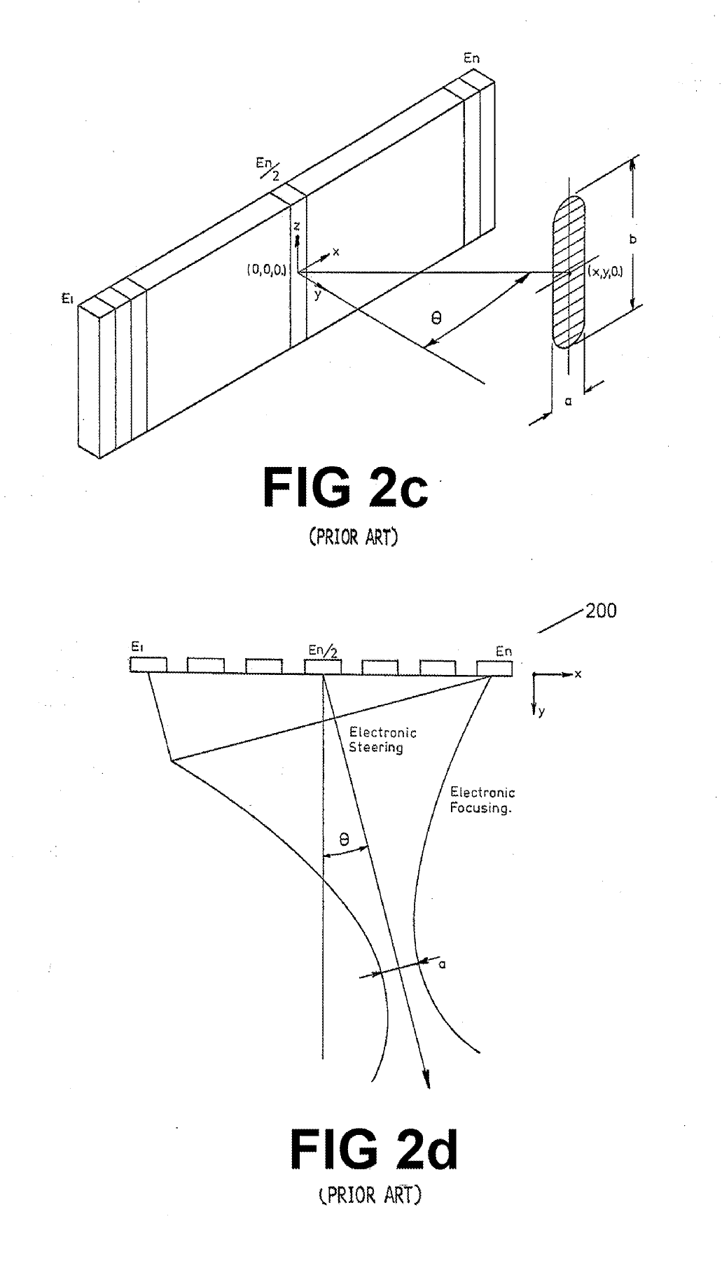

The present invention FIG. 1a illustrates a system 100 for gathering and transmitting ultrasonic imaging data for providing medical treatment at a remote location, specifically acquiring and transmitting ultrasonic diagnostic image information. The system 100 includes an ultrasonic device 200 comprising the ultrasonic receiver/transmitter, which is fixed in place by an upper containment plate 17, such as a plastic structure. Said ultrasonic device 200, with or without said containment plate 17, may be contained within a glove 12 as shown in FIG. 1b. In one embodiment, the glove member 12 contains interface unit 524 that communicates via a transceiver or via a cable 124 with unit 524. Interface unit 524 may be used to display the ultrasonic images locally or may be used to transmit diagnostic signals, or information, from the medical diagnostic ultrasonic interface unit to a remote site 155. In one embodiment the interface unit 524 does not have a capacity for directly displaying the ultrasonic images at the patient site, but among other things, assists in the transmission of the images to the remote site 155 through a remote interface unit 524a. In a preferred embodiment, the interface units 524, 524a communicate with each other via a LAN/WAN Internet connection 50, including one of a telephone, satellite or radio transmission. The interface units 524, 524a can alternatively communicate to respective computers, such as a personal computer 156, 156a, tablet or intelligent phone 154, 154a or mainframe computer 170, having a database 112 attached thereto, and communicate thru remote interface unit 524a with a personal 80.

U.S. Pat. No. 6,248,064, incorporated herein by reference, generally describes the physical characteristics of the glove depicted in FIG. 1b, wherein glove is comprised of a first layer and a second layer optionally made of a highly flexible natural or synthetic material, such as cotton flocked nitrile. Referring again to FIG. 1b, the first glove member 12 includes a palm portion (not shown) where the ultrasonic probe 200 is situated. The glove palm portion, in one non-limiting embodiment, exposes the surface of the ultrasonic probe elements, so as to apply an ultrasound gel or contacting agent between the probe and the patient's body to insure that there is no air gap since and acoustic signal is attenuated in the presence of such gap. Therefore, the probe will have an intimate contact with the patient's skin, physically and as through various surface contact products well known to those in the medical arts that currently administer ultrasonic imaging.

Referring to FIG. 1a, in one embodiment of the disclosure, a remote location 155 command center may provide remote control of the system 100. FIG. 1b, illustrates the glove 12, having incorporated therein an interface unit 524 for local timing and control functions of the ultrasonic probe array 200, utilizing special purpose electronic systems operatively under the control of the ultrasonic system 500 having a microprocessor 501 and associated RAM and ROM memory (not shown), executing control routines stored in memory. As will be discussed in detail below, the control program includes a variety of routines that modify the operation of the ultrasonic receiver 510 to produce a 3D image.

The ultrasonic glove 12 communicates with interface unit 524, which transmits image data to the remote location 155 command center for evaluation by diagnostician 80. The frame rate of the transmission may be adjusted by either the technician 20 or the diagnostician 80 depending upon the particular interest or application and the currently available communication devices and telephone or computer network bandwidth and quality of service. The frame rate may vary during a particular consultation in combination with increased or decreased resolution based on the current use of the glove 12. For example, a higher frame rate and lower resolution may be used for general conversation between patient 25, technician 20 and medical diagnostician 80 at remote location 155, while a higher resolution and lower frame rate or still picture may be used to provide a more detailed image or close-up of body portion of patient 25 to provide a more thorough visual examination.

Depending upon the particular application, the ultrasonic imaging system included in glove 12 may be remotely controlled by the medical diagnostician 80 to accentuate features to facilitate visual examination. For example, the ultrasonic system 500 may include remotely controllable focus, zone acquisition, pan, zoom, and off-axis controls that can be operated from remote location 155. In one embodiment the remote diagnostician 80 may direct the ultrasonic imaging beam over one zone to more closely investigate an anatomical or organic anomaly.

In one embodiment the glove 12 electronics is powered by a battery or other power source, and the containment unit 17 having the ultrasonic receiver/transmitter 200 therein, or optionally the glove 12 is directly affixed to the body of patient 25 using a strap or adhesive, such that the patient 25 may be ambulatory, that is walk, while the glove 12 is attached, permitting the electronics in the ultrasonic system 500 to transmit images from the ultrasonic array 200 to the local controls in interface unit 524 or directly via unit 524a at the remote location 155 to be viewed and controlled by diagnostician 80. In this way, a patient 25 may be outfitted with the ultrasonic imaging device incorporated in containment unit 17 or glove 12 and be monitored locally or remotely, and while ambulatory.

Ultrasonic glove 12 also preferably includes local controls for operation by the technician 20 or the patient 25 or others in response to requests or instructions from diagnostician 80. The glove 12 is preferably connected via a wired or wireless communication link 124a to interface unit 524, which may include appropriate electronic image processing and compression utilizing hardware and software. Alternatively, ultrasonic glove 12 may be connected to or integrated with local network device (not shown), which may include video processing hardware and/or software to process and/or store one or more video images. The remote location 155 further includes computer devices 170, 154a and 156a that are programmed to remotely control one of focus, zone acquisition, pan, zoom, and off-axis of the acoustic beam used to image the anatomy.

As disclosed in U.S. Pat. Nos. 7,860,725, 6,595,918, 6,540,673, 6,248,064, 6,224,548, 6,224,548, all hereby incorporated hereby by reference in their entirety, the glove 12 also may include other diagnostic devices, such as EKGs, temperature sensing, video monitoring and at least one microphone to provide an audio link from patient 25 or local surroundings. These devices, such as a microphone may be integrated into wearable device 12, interface unit 524, or network device.

As will be appreciated by those of ordinary skill in the art of electronics, the various signal conditioning, processing, and/or control functions required by processor function of ultrasonic system 500 and array 200 may be implemented solely through electronic circuitry using various fabrication techniques depending upon the desired form factor, manufacturing costs, and various other human factor and engineering considerations. However, these functions may also be performed by electronic circuitry in combination with software, firmware or one or more embedded microcontrollers and/or microprocessors having encoded logic and instructions stored in associated non-volatile memory to provide increased performance and flexibility while facilitating periodic enhancements or upgrades.

The present invention is independent of the particular implementation of the signal conditioning or processing circuitry, but contemplates a unified, composite circuit board implementation such that in one embodiment the form factor is suitable such that the transmission portion of the processor 524 may be contained in a network device, such as a mobile telephone, tablet or hand-held computer which can transmit or within its architecture produce an ultrasonic image or sonogram.

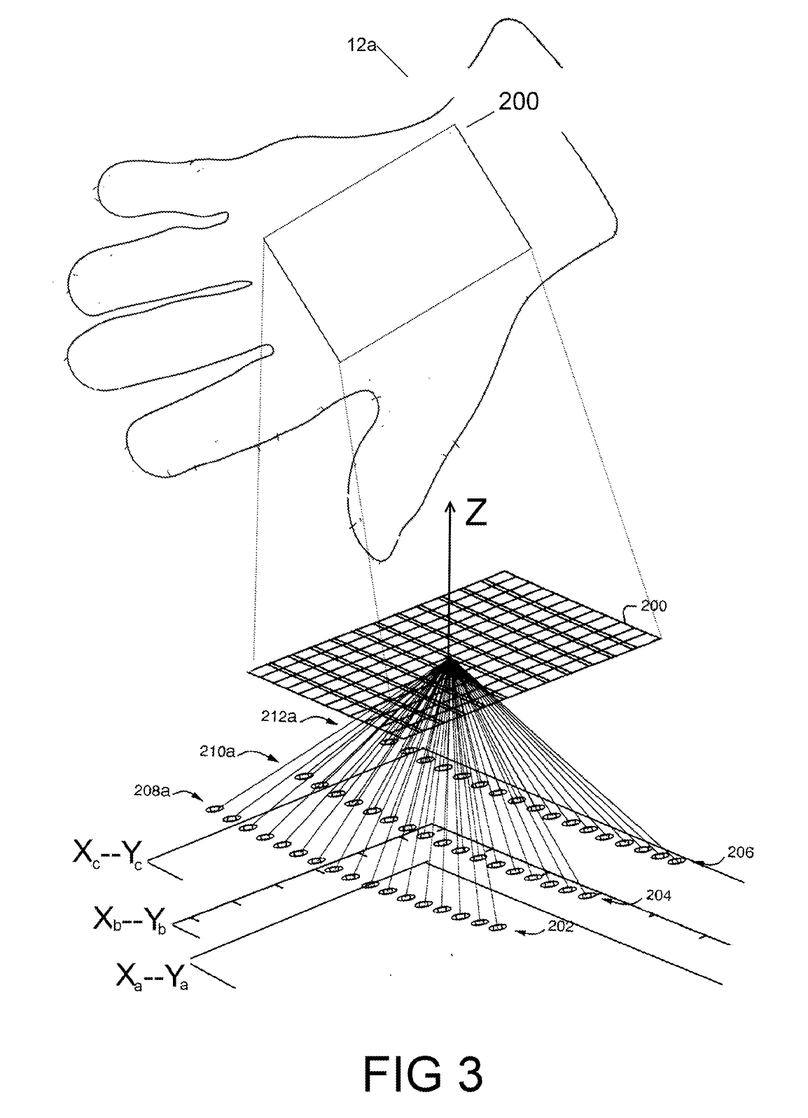

FIG. 1b shows glove 12, having incorporated therein a 2D array 200 under the control of the ultrasonic system 500 to produce a scan which results in a single frame of data obtained in accordance with a non-limiting embodiment of the invention. In one such non-limiting embodiment of the invention, FIG. 3 shows the array 200, which acquires data, in successive scans of three (3) X-Y planes: xₐ-yₐ, x_{b}-y_{b} and x_{c}-y_{b}, 202, 204, and 206, respectively. Each plane xₐ-yₐ, x_{b}-y_{b} and x_{c}-y_{b} comprises a series of scan lines 208a, 210a and 212, respectively running in the direction X. In this case, each plane is displaced in elevation along the Z-axis, from the neighboring full frame sweep.

To produce 3D images of a volume of ultrasonic data, a 3D scan data set must be created by scanning two or more planes and assembling the planes into a composite image displayed isometrically. One embodiment of the invention includes displaying a video composite image as one of an isometric image, colored features of the image, selected morphological features of the image and automatically recognized morphological features of the image. Scan data may be filtered or identified on the basis of the spatial zone from which it originated using among other variables, the amplitude of the received signal or the spatial or temporal proximity to other scan data and then used as a parameter to highlight a region such as an area, plane or zone from which it originated. Such highlighting may be achieved through associating a color with a highlighted region or outlining various perimeters of a region. The techniques of displaying of video composite images as isometric images, highlighting features and recognizing salient morphological features of these images are well understood by those of ordinary skill in the electronic arts. (See, K. Preston, Jr., “Feature Extraction by Golay Hexagonal Transforms,” IEEE Trans. Comput., September 1971; and K. Preston, Jr. and J. R. Carvalko, On Determining Optimum Simple Golay Marking Transforms for Binary Image Processing, IEEE Trans. Comput., December 1972; K. Preston, Jr. and J. R. Carvalko, “Use of the Contourograph to Evaluate a High-resolution Television Microscope,” Proceedings of the IEEE, January 1969.)

The scan lines of every sweep are typically X-Y across the array 200 “lateral” dimension as shown in FIG. 3. The planes of the successive sweeps in a frame are located with respect to each other, e.g. displaced along the Z-axis or in the “elevation” direction, which is typically at a 90 degree, right angle or orthogonal to the lateral X-Y dimension. Each scan frame comprises a plurality of lines in the X-direction allowing the assembly of a 3D scan data set representing a scan zone.

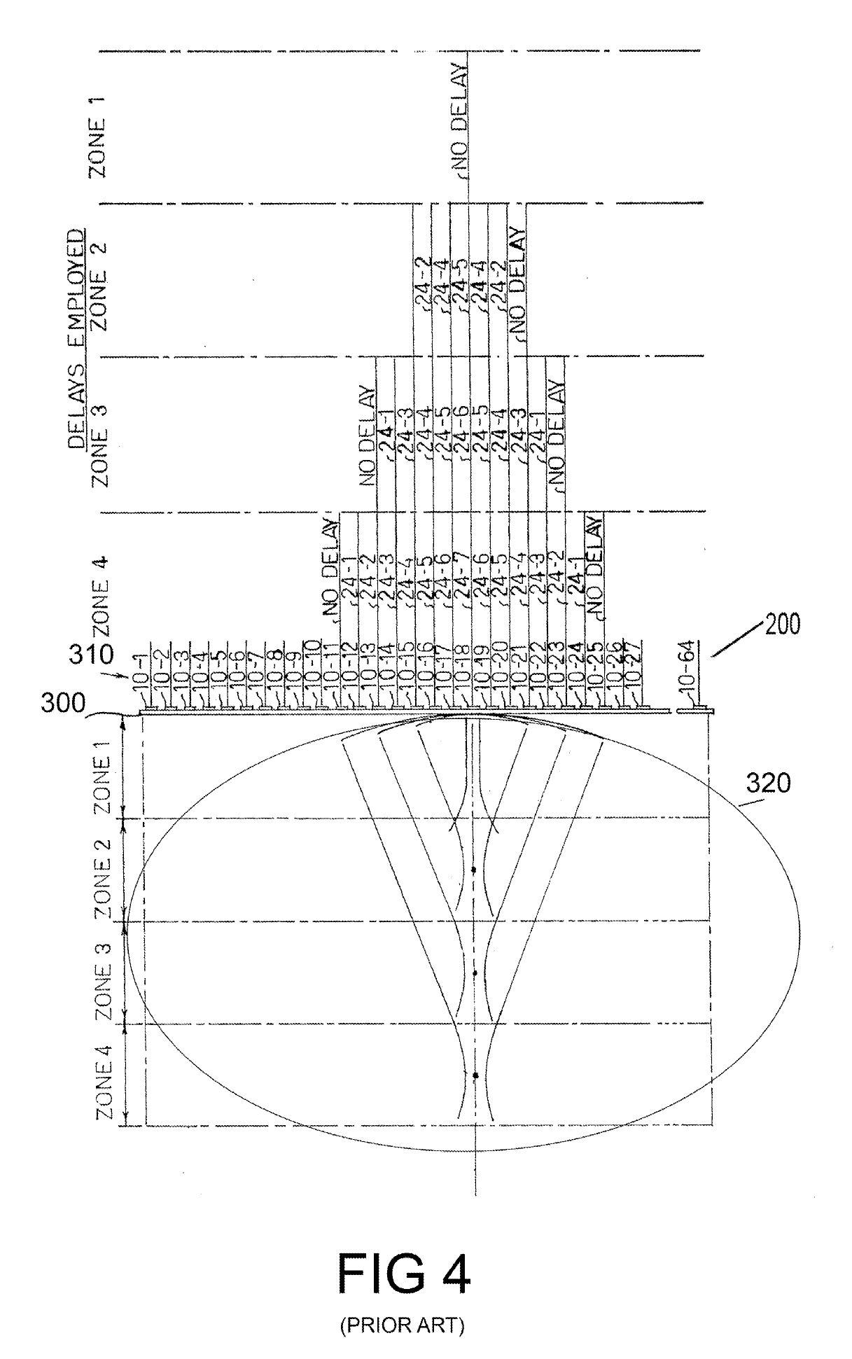

By way of describing a non-limiting embodiment of the present invention, FIG. 4 illustrates one line 310 of transducers 10-1 through 10-64 which are part of a two-dimensional acoustic array 300, which is in contact with the soft tissue of a body 320. By way of description and not limitation, the section to be imaged is shown divided into a plurality of four ranges of zones identified as zone 1 through zone 4 as depicted. The zones may be of equal or unequal width (i.e. range) as desired, with the number of range zones employed depending, among other things, upon desired resolution throughout the entire range of operation.

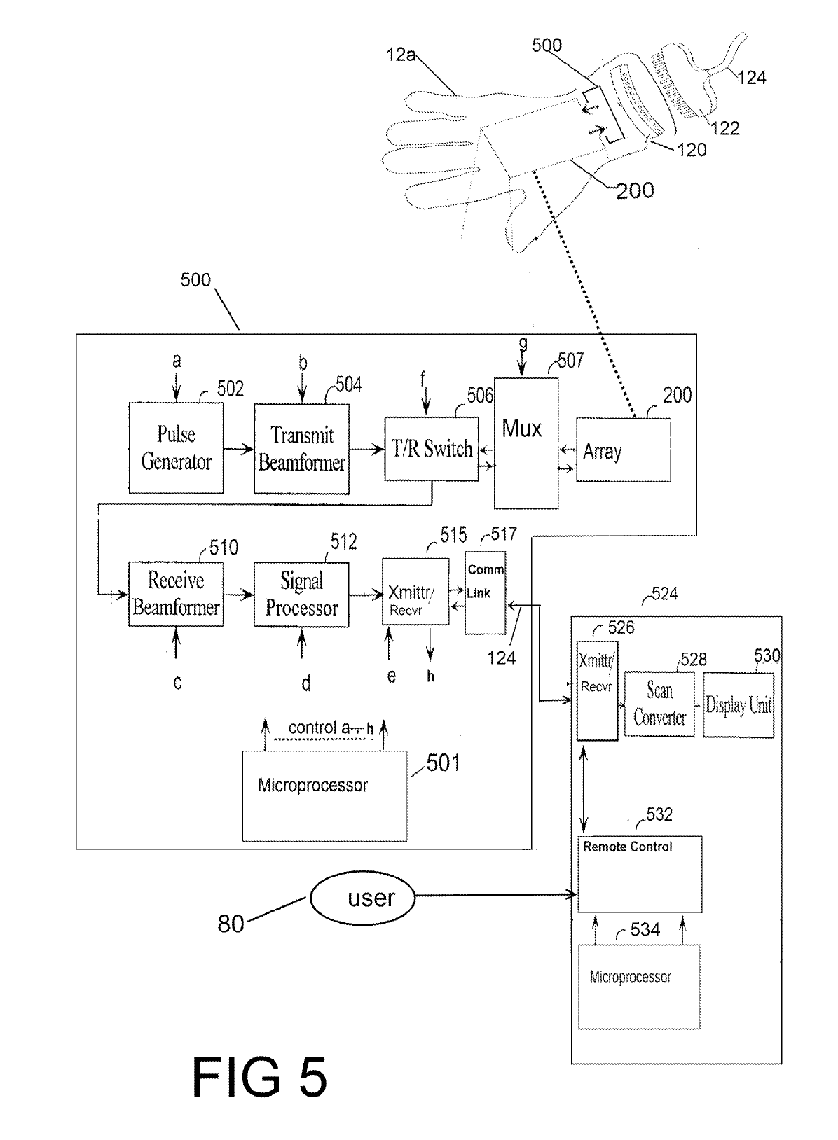

FIG. 5 is a simplified block diagram of the ultrasonic system 500 in accordance with a non-limiting embodiment of the present invention. Those of ordinary skill in the relevant art of electrical engineering know that the ultrasonic system 500 operation as described herein is intended to be representative and that any particular system may differ from that shown. Incorporated by reference herein are ultrasonic systems found in representative U.S. Patents as follows:

U.S. Pat. No. 6,669,641 B2 to Poland et al., entitled “Method of and System for Ultrasonic Imaging;” U.S. Pat. No. 6,641,534 B2 to Smith et al., entitled “Methods and Devices for Ultrasonic Scanning by Moving Sub-Apertures of Cylindrical Ultrasonic Transducer Arrays in Two Dimensions;” U.S. Pat. No. 6,491,634 B1 to Leavitt et al., entitled “Sub-Beamforming Apparatus and Method for a Portable Ultrasonic Imaging System;” U.S. Pat. No. 6,380,766 B2 to Savord, entitled “Integrated Circuitry for Use With Transducer Elements in an Imaging System;” U.S. Pat. No. 6,276,211 B1 to Smith, entitled “Methods and Systems for Selective Processing of Transmit Ultrasonic Beams to Display Views of Selected Slices of a Volume;” U.S. Pat. No. 6,122,223 to Hossack, entitled “Ultrasonic Transmit Waveform Generator;” U.S. Pat. No. 6,122,222 to Hossack et al., entitled “Ultrasonic Transmit and Receive System; “U.S. Pat. No. 6,179,780 B1 to Hossack et al., entitled “Method and Apparatus for Medical diagnostic Ultrasonic Real-Time 3-D Transmitting and Imaging;” U.S. Pat. No. 6,126,602 to Savord et al., entitled “Phased Array Acoustic Systems With Intra-Group Processors;” U.S. Pat. No. 6,013,032 to Savord, entitled “Beamforming Methods and Apparatus for Three-Dimensional Ultrasonic Imaging Using Two-Dimensional Transducer Array;” U.S. Pat. No. 5,997,479 to Savord et al., entitled “Phased Array Acoustic Systems With Intra-Group Processors;” U.S. Pat. No. 4,949,310 to Smith et al., entitled “Maltese Cross Processor: A High Speed Compound Acoustic Imaging System;” U.S. Pat. No. 5,933,389 to Hossack et al., entitled “Ultrasonic Imaging System and Method;” U.S. Pat. No. 5,619,999 to Von Behren et al., entitled “Body Surface Position Locator for Ultrasonic Transducer;” U.S. Pat. No. 5,566,675 to Li et al., entitled “Beamformer for Phase Aberration Correction;” U.S. Pat. No. 5,483,963 to Butler et al., entitled “Two Dimensional Transducer Integrated Circuit;” U.S. Pat. No. 5,447,158 to Nakajima et al., entitled “Ultrasonic Imaging Method and System Capable of Displaying B-Mode Image and Color Flow Mapping Image Over Wide Field;” U.S. Pat. No. 5,229,933, Larson, III, 2-D phased array ultrasound imaging system with distributed phasing; U.S. Pat. No. 5,063,541 to Kondo et al., entitled “Beam Forming Method and Apparatus Therefore in Ultrasonic Imaging System;” and U.S. Pat. No. 4,694,434 to von Ramm et al., entitled “Three-Dimensional Imaging System;” U.S. Pat. No. 4,305,296, Green , et al Ultrasonic imaging method and apparatus with electronic beam focusing and scanning; U.S. Pat. No. 3,936,791, Kossoff, Linear Array Ultrasonic Transducer.

By way of description and not limitation, referring now to FIG. 4 and FIG. 5, a suitable electrical signal delay circuit located in the transmit beamformer 504 utilizes seven delays 24-1 through 24-7. By way of example, and not limitation, during operation, the sets of individual delays ranging in number from zero to seven, are employed for focusing at the different range depth zones. For operation in zone 1, a suitable switching network such as multiplexer, mux 507 connects a selected one of the sixty-four transducer elements to a summing amplifier, via transmit-receive switch, without passage of the signal through any of the delay elements. The diagram of FIG. 4, under the heading “DELAYS EMPLOYED” indicates that no delay is employed for ZONE 1 operation. In FIG. 4, one line is shown leading from a single transducer element, here element 10-18, to the ZONE 1 section of the diagram to illustrate that for operation at ZONE 1 the transducer elements are operated singly (in sets of one). It further will be understood that during ZONE 1 operation the receiver is range-gated for processing signals received from the operative transducer element only during the time reflected signals are received from within the zone of interest.

Referring again to FIG. 5, under the control (e.g., process, timing and control functions) of a microprocessor 501, a pulse generator 502 applies pulse sequenced transmit signals to a transmit beamformer 504. When a T/R switch 506 is in the transmit mode, the array of transducers 200, having multiple elements as in FIG. 4, such as line of transducers 310 of transducers 10-1 through 10-64, generate respective ultrasonic pressure (acoustic) signals.

Conventional beamforming operations are applied for each firing of signals to shape the beam parameters of direction, focus, field of view, and depth into a zone of interest in a patient's body and upon reflection of the ultrasonic energy, from various structures and organs within the patient's body produces ultrasonic images. As is known in the art, by appropriately delaying the waveforms applied to each transducer element by the transmit beamformer 504, a focused ultrasonic beam may be configured to produce one focused beam, multiple transmit beams or may essentially remain unfocused depending on the beamforming approach being used.

The ultrasonic energy from a given point within the patient's body is received by the array 200 transducer elements at different times as illustrated by the zones in FIG. 4, and may be amplified, individually delayed (internal to the beamformer) or as programmed for the desired delay in the microprocessor 501 and controlled via microprocessor lines 501b or 501c to the transmit beamformer 504 or receiver beamformer 510, respectively. In the case of the receive return signals, the signals are accumulated (e.g., via a memory) by the receive beamformer 510 to provide a beamformer signal that represents the received ultrasonic targets, within a particular zone, along a desired receive line scan line at a selected depth. The receive beamformer 510 may be an analog beamformer or a digital beamformer having an analog-to-digital converter for converting the transducer signals to digital signals. In some technologies for reproduction of an acoustic image, the received signal is mixed with the original acoustic carrier or reference beam that is used for the transmission signal, and then appropriately processed, as for example in signal processor 512, using Fast Fourier Transforming techniques, to convert the wavefront image into an object space, or real image. (See, and Grubinskas, R. C., State of the Art Survey on Holography and Microwaves in Nondestructive Testing. Army Materials and Mechanics Research Center, September 1972, available through National Technical Information Services, U.S. Department of Commerce, Springfield, Va.; and Preston K. Jr., Advances in Acoustical Holography, Angew Chem Int Ed Engl. 1972, available through http://onlinelibrary.wiley.com/doi/10.1002/anie.197202791/abstract, last visited May 1, 2012). Those skilled in the art of designing acoustic imaging may select the appropriate form as a design consideration for converting the acoustic data into a viewable image, 3D image.

The processed signal from signal processor 510 feeds a bi-directional transmitter/receiver 515 that conditions the image data for transmission to a communications link 517 in the embodiment shown in FIG. 5. The image from communications link 517 is transmitted either via a transmission data line or via a wireless link (not shown) to unit 524 transmit/receiver 526. Unit 524 may be situated locally where the patient is being examined or it may be situated remotely, such that a communications network, as shown in FIG. 1, reference 50, is employed. In this latter case, the link 124 is a wireless link that connects communications link 517 to the transmit/receive unit 524. When the image is sent to a scan converter 528 that allows a user to move through various slices of the zone (Z-axis) under investigation before it is displayed on display unit 530. Transmit/receiver 526 also is operatively connected to a remote control 532 that receives data concerning the topographic region and the acoustical-optical parameters, existing during the scan, as for example the X-Y coordinates, zone, focus, depth of field and frame rate. The user, typically diagnostician 80 may then input into the remote control 532, through a keyboard, mouse and browser, or hand control unit, the parameters that the diagnostician desires to use on subsequent scans.

The process of scanning a line such as in the X-axis direction is repeated for multiple scan lines along the Y-axis direction to create a frame of data for generating an image of the zone of interest in the patient's body. The receive beamformer 510 may, for example, be a beamformer of the type used in the model AGILENT SONOS 5500 ultrasonic system manufactured and sold by AGILENT TECHNOLOGIES.

In one embodiment the receive beamformer signals 510 are applied to a signal processor 512 to improve signal quality before the signal is supplied to a scan converter 514, which converts sector scan or other scan pattern signals to conventional raster scan display signals.

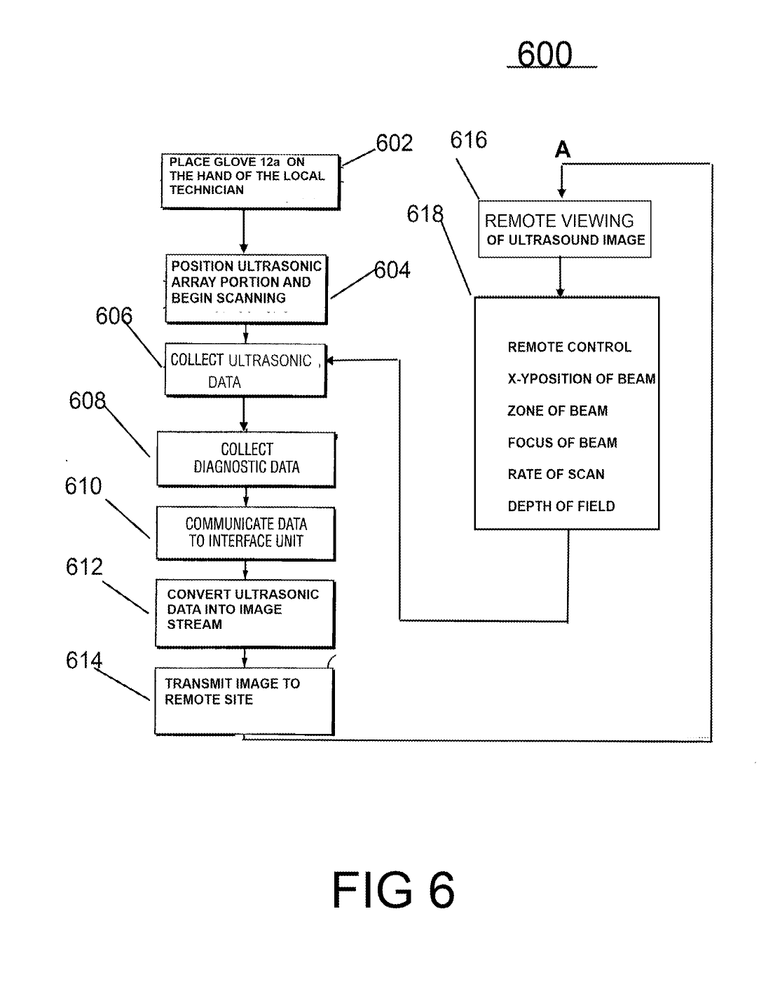

Turning to FIG. 6, one embodiment of the present invention is a method 600, which allows the remote control of the ultrasonic imaging of the scanner array 200. The method includes: fixing a wearable ultrasonic two-dimensional array over a part of a patient anatomy, electronically scanning the anatomy using the array to create a three-dimensional representation of the part of the anatomy, transmitting the data, converting the transmitted data to an image of the anatomy.

More specifically, diagnosing a medical condition is achieved by positioning the ultrasonic scanning device, such as glove 12 having the ultrasonic array contained therein, over a part of the patient 25 anatomy, scanning the anatomy ultrasonically, collecting ultrasonic image data, transmitting the data, viewing the images transmitted, locally or remotely, initiating the acquisition a subsequent series of ultrasonic images based on the viewing, and transmitting control data to alter the ultrasonic scanning device state of: X and Y position, Z position beam width, beam focus, rate of frame scan and depth of field. More particularly, in step 602, the local technician 20 or patient 25 slips their hand into the glove 12 and in step 604 positions the ultrasonic scanner over a part of the anatomy that the diagnostician 80 desires to image. The diagnostician or the local technician 20 or patient 25 begins the scanning operation. Upon an electronically generated command, again, as by way of example and not limitation, initiated by the remote diagnostician 80 or the local technician 20 or patient 25, the ultrasonic system 500 begins to collect ultrasonic data. In step 608, the system may be programmed to collect other diagnostic data as provided for by the glove 12 and described in the related applications herein. In step 610, the data, both ultrasonic and diagnostic, is communicated to the local interface unit, such as interface unit 524, which converts the ultrasonic data into an image stream, having lines and frames in a format chosen by the systems designers. The image is transmitted in step 614, as digital packets of information containing image intensity, zone, specific location of image point, as referenced by pixels, lines and/or frames. Such technology is well known in the electronic art of gathering and transmitting digital or analog image data. At the remote viewing site, such as remote site 155, the diagnostician 80, views the images at step 616, and decides, and initiates through controls at the remote site, among other things, the acquisition of the next series of ultrasonic images, by communicating control data that results in, by way of example and not limitation, a state of: the X and Y positioning of the ultrasonic beam, the Z position or zone of the beam, the degree of focus, the rate of frame scan, and the depth of field. For patients that have the wearable device semi-permanently installed the user can set a time during which the ultrasonic scanning device scans the anatomy.

While the present invention has been described with reference to the illustrative embodiments, this description is not intended to be construed in a limiting sense. Various modifications of the illustrative embodiments, as well as other embodiments of the invention, will be apparent to those skilled in the art on reference to this description. It is expressly intended that all combinations of those elements that perform substantially the same function in substantially the same way to achieve the same results are within the scope of the invention. Substitutions of elements from one described embodiment to another are also fully intended and contemplated. It is therefore contemplated that the appended claims will cover any such modifications or embodiments as fall within the true scope of the invention.

We claim:

1. An apparatus for transmitting a sonogram to a remote location comprising: a wearable ultrasonic array of transducers positioned over a portion of the patient's body, said array transmission electronically steered and focused for creating a three dimensional representation of a portion of a patient's anatomy, said representation transmitted to a remote location.

2. The apparatus in claim 1 wherein assembled data from two or more scanned planes results in a composite image of the representation displayed isometrically.

3. The apparatus in claim 1 further includes a computer that remotely controls one of transmission focus, zone acquisition, pan, zoom, and off-axis.

4. An apparatus for transmitting a sonogram to a remote location comprising: an array of ultrasonic transducers contained in a wearable glove positioned over a portion of the patient's body, said array transmission electronically steered and focused for creating a three dimensional representation of a portion of a patient's anatomy, said representation transmitted to a remote medical location.

5. The apparatus in claim 4 wherein the array of ultrasonic transducers are arranged in two dimensions.

6. The apparatus in claim 5 wherein the array of ultrasonic transducers are operatively connected to a beamformer.

7. The apparatus in claim 6 wherein the beamformer controls firing of signals to shape one of the beam parameters consisting of: direction, focus, field of view, or depth within a zone of interest in said patient's body.

8. The apparatus in claim 6 wherein the beamformer operates to delay the waveforms applied to each transducer element.

9. The apparatus in claim 4 wherein the array of ultrasonic transducers are in contact with the patient's body through an intermediate contacting agent.

10. A method for remote medical diagnosing by medical personnel comprising: fixing a wearable ultrasonic two-dimensional array over a part of a patient anatomy, electronically scanning the anatomy using the array to create a three-dimensional representation of the part of the anatomy, transmitting the data, converting the transmitted data to an image of the anatomy.

11. The method of claim 10, further including initiating the acquisition a subsequent series of ultrasonic images based on the viewing.

12. The method of claim 10, further including transmitting control data to alter the ultrasonic scanning device state of one of: X, Y, or Z position.

13. The method of claim 10, further including setting a time during which the ultrasonic scanning device scans the anatomy.

14. The method of claim 10, further including transmitting control data to alter the ultrasonic scanning device state of one of: the local technician or patient slips their hand into the glove.

15. The method of claim 10, further including displaying of video composite images as one of isometric images, colored features of the images, selected morphological features of the images or automatically recognized morphological features of the images.

16. The method of claim 10, further including mounting the wearable ultrasonic scanning device on a patient for ambulatory operation.

17. The method of claim 10, further including transmitting control data to alter positioning the ultrasonic beam over a part of the anatomy that a diagnostician desires to image.

18. The method of claim 10, further including storing the three-dimensional representation data before transmitting the data representation of a part of the anatomy that a diagnostician desires to image.

19. The method of claim 10, further including transmitting control data to alter the beamwidth of the ultrasonic beam over a part of the anatomy that a diagnostician desires to image.

20. The method of claim 10, further including transmitting control data to alter depth of field of the ultrasonic beam over a part of the anatomy that a diagnostician desires to image.
